(19) United States
(12) Patent Application Publication
(10) Pub. No.: US 2003/0235894 A1
(43) Pub. Date: Dec. 25, 2003

(54) Biological tagatose production by recombinant Escherichia coli

(75) Inventors: Pil Kim, Seoul (KR); Hoe Jin Roh, Incheon (KR); Sang Hyun Yoon, Seoul (KR); Jin Hwan Choi, Seoul (KR)

Correspondence Address: JACOBSON HOLMAN PLLC, 400 SEVENTH STREET N.W., SUITE 600, WASHINGTON, DC, 20004, US

(73) Assignee: Tongyang Confectioner Co.

(21) Appl. No.: 10/440,407

(22) Filed: May 19, 2003

Related U.S. Application Data
(63) Continuation of application No. 09/743,196, filed on Jan. 8, 2001, now abandoned.
Application No. 09/743,196 is a 371 of international application No. PCT/KR99/00661, filed on Nov. 4, 1999.

(30) Foreign Application Priority Data
May 6, 1999 (KR) 1999/16118

(51) Int. Cl. C12P 21/02; C12N 1/21; C12P 19/04; C12N 9/24; C12N 15/74

(52) U.S. Cl. 435/101; 536/123; 435/69.1; 435/252.33; 435/200; 435/320.1

(57) ABSTRACT

This invention relates to a recombinant Escherichia coli and a process for producing D-tagatose. In detail, it includes the construction of recombinant E.coli harboring L-arabinose isomerase, whole-cell conversion of D-galactose into D-tagatose by recombinant E.coli expressing L-arabinose isomerase, enzymatic production of D-tagatose by the extract of recombinant E.coli expressing L-arabinose isomerase, and bioconversion by immobilized L-arabinose isomerase.

### TECHNICAL FIELD

The present invention relates to a novel recombinant E. coli, a fermentation process and a enzymatic conversion process for tagatose production using such recombinant E.coli harboring vector containing the gene of L-arabinose isomerase, and the promoter controlled artificially.

### BACKGROUND ART

D-Tagatose is one of ketohexoses as well as one of D-galactose isomers. D-Tagatose is reported as the sweetener having the taste most similar to that of sucrose (92%). In addition, D-tagatose does not show laxative effect, which other polyols generally do. Due to such reasons, D-tagatose has been recently regarded as a non-caloric sweetener substituted by sucrose [Zehener, L. R., D-Tagatose as a low-calorie carbohydrate sugar and bulking agent, EP 257626 (1988) ; Marzur, A. W., Functional sugar substitutes with reduced calories, EP 341062 (1989)].

Several methods have been studied for the manufacture of D-tagatose. D-Galactose could be converted into D-tagatose in the presence of a calcium catalyst [Beadle, J. R., Saunders, J. P., and Wajda, T. J., Process for manufacturing tagatose, WO 92/12263 (1992)]. Although the chemical synthesis is economical, this process also requires disadvantageous high temperature and high pressure. Recently, the biological process has been researched with interest as an environmentally clean process. As a consequence, the biological production of D-tagatose has been intensively studied recently.

Among the such studies, D-tagatose production from galactitol using galactitol dehydrogenase has been well known [Izumori, K., and Keiji, T., “Production of D-tagatose from D-galactitol by Mycobacterium smegmatis”, J. Ferment. Technol., 15, 105-108 (1988); Shimonish, T., Okumura, Y., Izumori, K. “Production of L-tagatose from galactitol by Klebsiella pneumoniae strain 40b”, J. Ferment. Bioeng., 79, 620-622 (1995)]. Galactitol, however, is more expensive than galactose and seems to be that the commercial application of galactitol is only a little potential.

It has been suggested that L-arabinose isomerase (AraA) could convert galactose into tagatose. Cheetham reported that L-arabinose isomerase from Mycobacterium and Lactobacillus catalyzes the conversion from D-galactose to D-tagatose as well as that of from L-arabinose to L-ribulose, because of the similar substrate configuration [Cheetham, P. S. J., and Wootton, A. N., “Bioconversion of D-galactose into D-tagatose”, Enzyme Microbiol. Technol., 15, 105-108 (1993)]. Enterobacter agglomerans could also produce tagatose from galactose during the growth on the arabinose pre-induced medium. Such implies the fact that arabinose isomerase could mediate the conversion of tagatose [Kim, S. Y., Roh, H. J., and Oh, D. K., “D-Tagatose production from D-galactose by Enterobacter agglomerans TY-25”, Kor. J. Appl. Microbiol. Biotechnol., 25, 490-494 (1997)].

### DISCLOSURE OF INVENTION

The object of the present invention is to provide a recombinant E.coli having a new metabolic pathway for producing tagatose from galactose.

Another object of the present invention is to provide new method for tagatose production by using said recombinant E.coli.

The further object of the present invention is to provide a new method for tagatose production by using the L-arabinose isomerase originated from said recombinant E.coli.

Further, the present invention relates to a recombinant E.coli (KCTC-0603BP) harboring vector comprising i) the gene of L-arabinose isomerase (EC 5.3.1.4; araA) and ii) the promoter controlled artificially (pTC101) for tagatose production. In said recombinant E.coli, araA is originated from the group consisting of E.coli, Bacillus, Salmonella, Enterobacter, Klebsiella, Pseudomonas,l Lactobacillus, Zymononas, Gluconobacter, Rhizobium, Acetobacter, Rhodobacter, Agrobacterium, and other microorganisms. Further, araA is integrated into the host chromosome.

The another aspect of the present invention relates to a fermentation process for tagatose production wherein the medium comprises 10-300 g/L of galactose, 7-13 g/L of yeast extract, 2-4 g/L of KH₂PO₄, 5-7 g/L of Na₂HPO₄, and 1-3 g/L of ammonium chloride. Further, L-arabinose isomerase from said strain mediates galactose to tagatose conversion. On the other hand, the L-arabinose isomerase can be immobilized for the recycle. At this time, the conversion medium comprises 10-500 g/L of galactose, 24 g/L of KH₂PO₄, and 5-7 g/L of Na₂HPO₄.

### BRIEF DESCRIPTION OF THE DRAWINGS

FIG. 1 is a chemical structure of D-tagatose and D-galactose.

FIG. 2 is a schematic diagram of bioconversion by L-arabinose isomerase.

FIG. 3 is a photograph which represents PCR product of L-arabinose isomerase from E.coli. Arrow indicates the PCR product of Ara A from E.coli (1.5 kb). Lane 1 shows the size marker (1 kb DNA Ladder, NEB, USA).

FIG. 4 is a plasmid map of pTC101, which harboring L-arabinose isomerase.

FIG. 5 is a photograph which represents EcoRI-HindIII double digest of pTC101. Arrow indicates the pTC101. Lane 1 shows the size marker (1 kb DNA Ladder, NEB, USA).

FIG. 6 is a profile of tagatose production by purified L-arabinose isomerase.

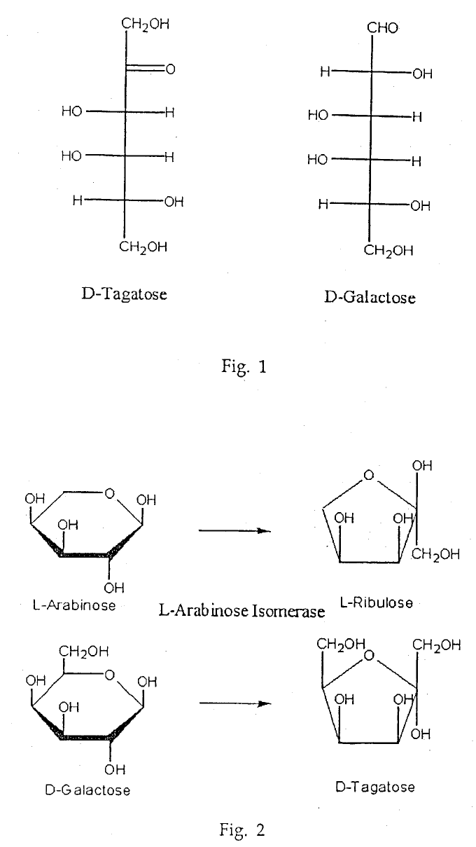

### BEST MODE FOR CARRYING OUT THE INVENTION

D-Tagatose is a potential bulking agent in food as a non-caloric sweetener. To produce D-tagatose from cheaper resources, plasmids harboring the L-arabinose isomerase gene (araA) from Escherichia coli is constructed because L-arabinose isomerase has been suggested as an enzyme that mediates the bioconversion of galactose to tagatose as well as that of arabinose to ribulose. The constructed plasmids has been named as pTC101, which contains the araA from E.coli. During the cultivation of recombinant E.coli with pTC101, tagatose has been produced from galactose in 9.0-11.0% yields.

The purified L-arabinose isomerase of E.coli with the plasmid pTC101 has also produced 25˜35 g/L of tagatose from 100 g/L of galactose for 150˜180 hours bioconversion. The enzyme extract of E.coli with the plasmid pTC101 has been immobilized into alginate bead. By using an immobilized enzyme system, 11˜14 g/L of tagatose has been produced from 10% galactose for 24 hours.

This invention includes the application of recombinant E.coli. The characteristics of E.coli are as follows. E.coli is a Gram negative facultative anaerobic rod-form bacteria [Holt J. G., et. al., Bergey's Manual of Systematic Bacteriology 9th ed., 179, Williams & Wilkins (1994)]. It shows simple nutrient requirement, fast life cycle (about 20 min) in the optimal condition (pH 7.0, 37° C.) [Stanier R. Y., et. al., The Microbial World, 5th ed., 439˜452(1986)].

In addition to the above characteristics, E.coli is the most well known organism in physiology and genetics. Therefore, metabolism of E.coli can be easily controlled by the methods of genetic engineering and metabolic engineering.

There are no direct evidences about the enzyme involving in the isomerization reaction of galactose to tagatose in E.coli. Kim et al, however, reported that Enterobacter induced by arabinose produces tagatose from galactose substrate [Kim, S. Y., Roh, H. J., and Oh, D. K., D-Tagatose Production from Galactose by Enterobacter agglomerans TY-25, Kor. J. Appl. Microbiol. Biotechnol., 25, 490-494 (1997)], which implicates that some enzyme(s) induced by arabinose can convert galactose into tagatose. Cheetham and Wootton suggested that arabinose isomerase of Lactobacillus had a potential to convert galactose into tagatose [Cheetham and Wootton., Bioconversion of D-galactose into D-tagatose, Enzyme Microbiol. Technol., 15, 105-108 (1993)].

Based on the above reports, the gene of L-arabinose isomerase (araA) of E.coli W3110 has been cloned by using a PCR technique, inserted into vector pKK223-3, which yields pTC101. The constructed plasmids harboring araA from E.coli has been used to transform E.coli JM105 (E.coli JM105/pTC101). For the expression of AraA, 1 mM IPTG was added to medium.

L-Arabinose isomerase (AraA) is an enzyme mediating the conversion of arabinose to ribulose, which is induced by arabinose and inhibited by glucose. It have been suggested AraA could mediate galactose to tagatose with low efficiency because of the substrate similarity between arabinose and galactose (FIG. 2) [Cheetham and Wootton., Bioconversion of D-galactose into D-tagatose, Enzyme Microb. Technol., 15, 105-108 (1993)].

The normal E.coli does not express AraA in the medium for tagatose production because the medium contains galactose as a sole carbon source. In the case of E.coli JM105/pTC101, the AraA could be artificially induced by adding an IPTG.

Said E.coli JM105/pTC101 was deposited in Korean Collection for Type Cultures in Korea Research Institute of Bioscience and Bioengineering, in accession number of KCTC-0603BP on Apr. 26, 1999 under Budapest Treaty.

The present invention will be more specifically explained by the following examples. However, it should be understood that the examples are intended to illustrate but not in any manner to limit the scope of the present invention.

### EXAMPLE 1

Construction of Recombinant E.coli Harboring Arabinose Isomerase Gene

Construction of recombinant E.coli was followed by Sambrook method Sambrook et al., Molecular Cloning: A Laboratory Manual 2nd Ed., Cold Spring Harbor Laboratory Press (1989)].

Step 1 PCR cloning of araA and Insertion into Expression Vector

The template for PCR of araA was chromosome of a wild type E.coli W3110 [Genetic stock center collection number(CGSC) 4474]. The each primer used in PCR contained the part of araA terminal sequence and restriction enzyme site (EcoRI and HindIII, respectively).

| | | |
|---|---|---|
| FORWARD | 5′-GACGAATTCATGACGATT-3′ | (SEQ ID NO. 1) |
| BACKWARD | 5′-TGCAAGCTTTTAGCGACG-3′ | (SEQ ID NO. 2) |

As the above result, 1.5 kb of DNA fragment was obtained (FIG. 3). The obtained PCR product (1.5 kb) was ligated into expression vector, pKK223-3 (4.5 kb, Pharmacia, Uppsala, Sweden), after doulbe digest by EcoRI-HindIII restriction enzyme. The PCR product sequence (SEQ ID NO. 3) is as follows.

| | |
|---|---|
| atgacgattt ttgataatta tgaagtgtgg tttgtcattg gcagccagca tctgtatggc | 60 |
| ccggaaaccc tgcgtcaggt cacccaacat gccgagcacg tcgttaatgc gctgaatacg | 120 |
| gaagcgaaac tgccctgcaa actggtgttg aaaccgctgg gcaccacgcc ggatgaaatc | 180 |
| accgctattt gccgcgacgc gaattacgac gatcgttgcg ctggtctggt ggtgtggctg | 240 |
| cacaccttct ccccggccaa aatgtggatc aacggcctga ccatgctcaa caaaccgttg | 300 |
| ctgcaattcc acacccagtt caacgcggcg ctgccgtggg acagtatcga tatggacttt | 360 |
| atgaacctga accagactgc acatggcggt cgcgagttcg gcttcattgg cgcgcgtatg | 420 |
| cgtcagcaac atgccgtggt taccggtcac tggcaggata aacaagccca tgagcgtatc | 480 |
| ggctcctgga tgcgtcaggc ggtctctaaa caggataccc gtcatctgaa agtctgccga | 540 |
| tttggcgata acatgcgtga agtggcggtc accgatggcg ataaagttgc cgcacagatc | 600 |
| aagttcggtt tctccgtcaa tacctgggcg gttggcgatc tggtgcaggt ggtgaactcc | 660 |
| atcagcgacg gcgatgttaa cgcgctggtc gatgagtacg aaagctgcta caccatgacg | 720 |
| cctgccacac aaatccacgg caaaaaacga cagaacgtgc tggaagcggc gcgtattgag | 780 |
| ctggggatga agcgtttcct ggaacaaggt ggcttccacg cgttcaccac cacctttgaa | 840 |
| gatttgcacg gtctgaaaca gcttcctggt ctggccgtac agcgtctgat gcagcagggt | 900 |
| tacggctttg cgggcgaagg cgactggaaa actgccgccc tgcttcgcat catgaaggtg | 960 |
| atgtcaaccg gtctgcaggg cggcacctcc tttatggagg actacaccta tcacttcgag | 1020 |
| aaaggtaatg acctggtgct cggctcccat atgctggaag tctgcccgtc gatcgccgca | 1080 |
| gaagagaaac cgatcctcga cgttcagcat ctcggtattg gtggtaagga cgatcctgcc | 1140 |
| cgcctgatct tcaataccca aaccggccca gcgattgtcg ccagcttgat tgatctcggc | 1200 |
| gatcgttacc gtctactggt taactgcatc gacacggtga aaacaccgca ctccctgccg | 1260 |
| aaactgccgg tggcgaatgc gctgtggaaa gcgcaaccgg atctgccaac tgcttccgaa | 1320 |
| gcgtggatcc tcgctggtgg cgcgcaccat accgtcttca gccatgcact gaacctcaac | 1380 |
| gatatgcgcc aattcgccga gatgcacgac attgaaatca cggtgattga taacgacaca | 1440 |
| cgcctgccag cgtttaaaga cgcgctgcgc tggaacgaag tgtattacgg gtttcgtcgc | 1500 |
| taa 1503 | |

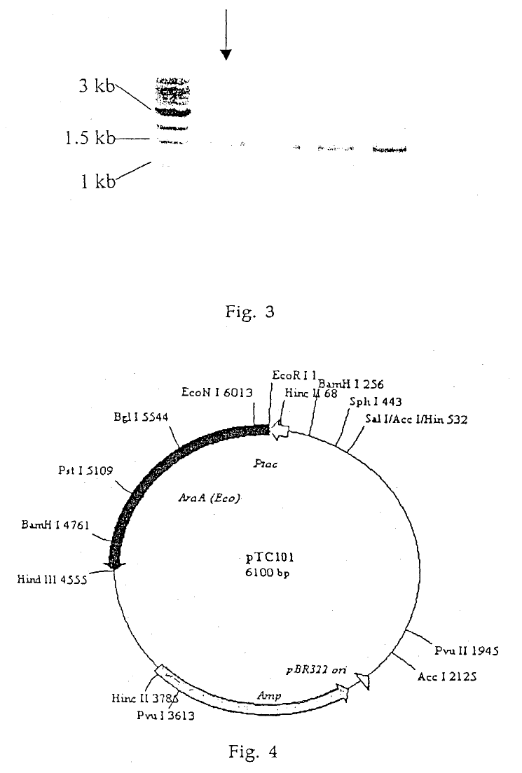

The constructed plasmid, named pTC101, was transformed into E.coli JM105 [Roh, H. J., Kim, P., Park Y. C., and Choi, J. H., Biotechnol. and Appl. Biochem., BA99/65, in press] (FIG. 4).

Step 2 Strain Selection

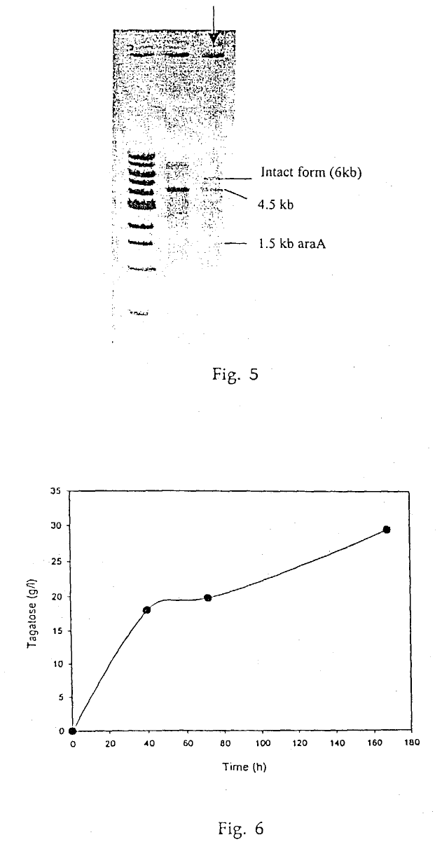

The transformed strains were selected on the plate containing ampicillin (50 g/ml) on the base of ampicillin-resistance. The colonies were further confirmed by restriction enzyme which digests the harbored plasmids, which shows 1.5 kb+4.5 kb DNA fragments at the time of digestion by EcoRI and HindIII (FIG. 5). The finally selected strain was named as E.coli JM105/pCT101, and said E.coli JM105/pTC101 was deposited in Korean Collection for Type Cultures in Korea Research Institute of Bioscience and Bioengineering, in accession number of KCTC-0603BP on Apr. 26, 1999 under Budapest Treaty.

### EXAMPLE 2

Tagatose Production by the Whole Cell of Recombinant E.coli

The E.coli JM105/pTC100 was pre-cultured in the LB-medium, and then transferred into main culture medium. The main medium contained following components (Table 1).

TABLE 1

| | | | | | Yeast |
|---|---|---|---|---|---|
| | Galactose | KH₂PO₄ | Na₂HPO₄ | NH₄Cl | extract |
| Concentration | 20 | 6 | 3 | 1 | 10 |
| (g/L) | | | | | |

Single colony of E.coli JM105/pTC101 was inoculated into 3 ml LB medium, and overnight cultured at 37° C. aerobically. The preculture was transferred into 250 ml Erlenmyer flask containing 50 ml of the IPTG (1 mM) in LB medium. When biomass reached absorbance 1.0 at 600 nm, cells were collected by centrifuge and inoculated into 3 ml of main medium. The main culture was maintained for 96 hrs at 37° C., 250 rpm shaking incubator. Finally, 0.86 g/L of tagatose was produced from recombinant E.coli whole cell, where no tagatose was found in the wild type E.coli. (Table 2).

TABLE 2

| | Tagatose Produced | Galactose Remained |
|---|---|---|
| Strain | (g/L) | (g/L) |
| JM105 | 0 | 10.0 |
| JM105/pTC101 | 0.86 | 12.2 |
| (KCTC-0603BP) | | |

Tagatose and galactose were estimated by using a HPLC (alliance 2690, Waters, USA) with RI detector (RID410, Waters, USA). The separation was made by C18-amine column (KR100-10 NH₂, Kromasil, Bohus, Sweden) eluted isocratically by 80% acetonitrile (35° C., 2 ml/min).

As shown in Table 2, recombinant E.coli expressing L-arabinose isomerase (KCTC-0603BP) could convert galactose into tagatose, where control strain could not.

### EXAMPLE 3

Tagatose Production by Crude Extract of Recombinant E.coli Expressing AraA

A crude extract of AraA from E.coli was prepared as follows. Actively growing cells (50 ml, O.D. 600 2.0) in the LB-medium containing IPTG (1 mM) were prepared by centrifugation (7,000 g, 4° C.). The cell pellet was resuspended in a 5 ml of phosphate buffer (50 mM, pH 7.0). The cells were disrupted by using an ultrasonic processor (50-watt Model, Cole-Parmer) at 40% output for 10 min in the ice. The crude arabinose isomerase solution was obtained after removal of cell-debris by centrifugation of the cell-disrupted suspension at 7,000 g and 4° C. for 15 min. A crude extract of AraA was fractionated by ammonium sulfate salting out at 40-60% saturation, and pelleted by centrifuge (10,000g, 4° C.). The pellet was further dialyzed overnight at 4° C. using a cellulose membrane tubing (MWCO: 12,000, Sigma, USA) to remove ammonium sulfate. Column chromatography was further carried out using DEAE (Sigma, USA) and Q-Sepharose (Sigma, USA) as anion exchange resins. The final purified L-arabinose isomerase showed 43 U/ml, where one unit defined as the amount of enzyme which produced 1 g-tagatose per minute at 35° C., pH 7.0.

The reaction mixtures consisted of 9 ml of galactose (1 g) dissolved in 50 mM phosphate buffer (pH 7.0) and 1 ml of enzyme solution mentioned the above. The bioconversion for tagatose production was performed at 35° C., pH 7.0 for 168 hrs. As shown in FIG. 6, the final tagatose concentration was reached 29.5 g/L. Remained galactose was 70.5 g/L.

### EXAMPLE 4

Tagatose Production by Immobilized Enzyme of L-Arabinose Isomerase

The purified L-arabinose isomerase as above was immobilized by a Calcium-Alginate. Briefly, 10 ml of the purified enzyme solution (381 U/ml) was mixed with the 10 ml of 0.15 M NaCl containing high-viscosity alginate gel (2.4%), then slowly expressed through a needle (id=0.3 mm) in a dropwise fashion into a 100 mM CaCl₂ solution. After instantaneous gelation, the beads were allowed to polymerize further for the period of 10 min in the CaCl₂ solution. The beads were added into 50 ml of galactose (100 g/L) dissolved in 50 mM phosphate buffer (pH=7.0). The conversion was performed for 24 hrs at 35° C. incubator. Table 3 shows the result.

TABLE 3

| | Free Enzyme | Immobilized Enzyme |
|---|---|---|
| Tagatose Production | 21.2 | 12.3 |
| (g/L) | | |

As shown in the Table 3, free enzyme solution gave 21.2 g/L where immobilized one gave 12.3 g/L of tagatose. This result indicates there is a mass transfer bottleneck in the case of immobilized enzyme whereas no mass transfer inhibition in the case of free enzyme. Though the immobilized enzyme showed low activity in the tagatose production, it could be recycled by simple filtration while free enzyme could not.

What is claimed is:

1. A recombinant E.coli (KCTC-0603BP) harboring vector comprising i) the gene of L-arabinose isomerase (EC 5.3.1.4; araA) and ii) the promoter controlled artificially (pTC101) for tagatose production.

2. The recombinant E.coli according to claim 1, wherein araA is originated from the group consisting of E.coli, Bacillus, Salmonella, Enterobacter, Klebsiella, Pseudomonas, Lactobacillus, Zymononas, Gluconobacter, Rhizobium, Acetobacter, Rhodobacter, Agrobacterium, and other microorganisms.

3. The recombinant E.coli according to claim 1, wherein araA is integrated into the host chromosome.

4. A fermentation process for tagatose production using the whole cell of recombinant E.coli of claim 1.

5. The fermentation process for tagatose production according to claim 4, wherein the medium comprises 10-300 g/L of galactose, 7-13 g/L of yeast extract, 24 g/L of KH₂PO₄, 5-7 g/L of Na₂HPO₄, and 1-3 g/L of ammonium chloride.

6. The enzymatic conversion process for tagatose production by L-arabinose isomerase, wherein the L-arabinose isomerase from strain of claim 1 mediates isomerization of galactose into tagatose.

7. The enzymatic conversion process for tagatose production according to claim 6, wherein the L-arabinose isomerase is immobilized for the recycle.

8. The enzymatic conversion process for tagatose production according to claim 6 or claim 7, wherein the conversion medium comprises 10-500 g/L of galactose, 24 g/L of KH₂PO₄, and 5-7 g/L of Na₂HPO₄.
